(19) United States
(12) Patent Application Publication
(10) Pub. No.: US 2026/0315114 A1
(43) Pub. Date: Oct. 15, 2026

(54) PEDICLE SCREW ASSEMBLY

(71) Applicant: CG BIO CO., LTD., Seoul (KR)

(72) Inventors: Ui Su JUNG, Seoul (KR); Samuel CHO, Englewood Cliffs, NJ (US)

(73) Assignee: CG BIO CO., LTD., Seoul (KR)

(21) Appl. No.: 19/485,265

(22) PCT Filed: May 14, 2024
(86) PCT No.: PCT/KR2024/006607

§ 371 Date: Nov. 17, 2025

(30) Foreign Application Priority Data
May 18, 2023 (KR) 10-2023-0064290
Dec. 12, 2023 (KR) 10-2023-0179868

(51) Int. Cl. A61B 17/70

(52) U.S. Cl. CPC A61B 17/7032; A61B 17/7035; A61B 17/7082

(57) ABSTRACT

Disclosed is a pedicle screw assembly. The pedicle screw assembly according to an aspect of the present invention includes: a screw member which extends in one direction and couples to a pedicle; and a separation prevention member for rotatably supporting the screw member. The screw member includes: a screw body which extends in the one direction and is at least partially inserted into the pedicle; a screw head positioned at one end of the extending direction of the screw body and coupled to the separation prevention member so as to be rotatable about the axis in the one direction; and a screw protrusion coupled to the screw head so as to face the screw body with the screw head interposed therebetween, and coupled to the separation prevention member so as to be rotatable about the axis in the one direction.

### CROSS-REFERENCE TO RELATED APPLICATIONS

The present application is a U.S. national stage application under 35 U.S.C. § 371 of International Patent Application No. PCT/KR2024/006607, filed May 14, 2024, which claims priority to and the benefit of Korean Patent Application No. 10-2023-0064290, filed on May 18, 2023, and Korean Patent Application No. 10-2023-0179868, filed on Dec. 12, 2023, all of which are incorporated herein by reference in their entireties.

### TECHNICAL FIELD

The present disclosure relates to a pedicle screw assembly, and more particularly, to a pedicle screw assembly having a structure in which a coupling length or angle can be easily adjusted without a separation process after being coupled to a spine.

### BACKGROUND

A pedicle screw, also referred to as a pedicle screw implant (hereinafter referred to as “pedicle screw”), is a device fixed to a spine. The pedicle screw is widely used to restore a deformed spine to its normal alignment. Specifically, after a plurality of pedicle screws are coupled to pedicles, a rod or similar component is inserted into the coupled pedicle screws to apply an external force, thereby allowing the spine to be restored back to its normal state.

The pedicle screws penetrate the skin and muscles covering the spine to access the spine. Therefore, in order to insert and fix the pedicle screw into the spine, an invasive procedure, that is, an incision at the surgical site, must be performed beforehand.

However, as the size of the invasive site increases, the difficulty of the surgery also rises, and the patient may experience greater pain. In addition, if the size of the invasive site becomes excessively large, there is an increased risk of secondary infections caused by bacteria and other pathogens.

Therefore, recently, various approaches have been actively discussed to minimize the size of the invasive site while enabling the insertion of the pedicle screw.

Meanwhile, after a plurality of pedicle screws are coupled to the spine, a configuration for applying an external force to the spine, such as a rod, needs to be coupled across the plurality of pedicle screws. In this case, when angles of some of the pedicle screws are misaligned, it becomes difficult to accurately couple the rod.

If the pedicle screw is not rotatable, a process of separating the pedicle screw already coupled to the spine and recoupling it to the spine while adjusting its angle needs to be performed. This causes additional pain to the patient and may also reduce the likelihood of a successful surgery.

Korean Patent No. 10-1252596 discloses a spine fixator. Specifically, it discloses a spine fixator including at least two pedicle screws and at least one connecting member interposed therebetween, the spine fixator including a pedicle screw that combines advantages of monoaxial and polyaxial screws.

However, in the spine fixator disclosed in the above prior art, a screw head is supported immovably within a screw shank to provide optimal restoration of the spine. That is, the spine fixator disclosed in the prior art does not provide a means for adjusting an angle without separating a pedicle screw already coupled to the spine.

Korean Patent No. 10-1149094 discloses a method for improving pedicle screw installation. Specifically, it discloses a method capable of installing a pedicle screw at an accurate position by precisely determining an incision site using a computer or the like, and inserting the pedicle screw into the determined incision site.

However, the method for improving pedicle screw installation disclosed in the above prior art is limited to securing an accurate site. The prior art does not provide a means for easily adjusting a coupling angle with a rod without separating a screw already coupled to the spine.
Korean Patent No. 10-1252596 (2013 Apr. 9.)
Korean Patent No. 10-1149094 (2012 Dec. 25.)

### SUMMARY

The present disclosure has been made to solve the above problems, and an object of the present disclosure is to provide a pedicle screw assembly having a structure capable of minimizing an invasive site.

Another object of the present disclosure is to provide a pedicle screw assembly having a structure capable of easily adjusting an angle while being coupled to the spine.

Yet another object of the present disclosure is to provide a pedicle screw assembly having a structure capable of easily adjusting an angle without a separation process while a screw is coupled to the spine.

Still another object of the present disclosure is to provide a pedicle screw assembly having a structure capable of changing a coupling position of a configuration connecting a plurality of screws without changing a coupling length of the screws.

An additional another object of the present disclosure is to provide a pedicle screw assembly having a structure capable of stably maintaining a coupling state between a screw coupled to the spine and another component.

The technical problems of the present disclosure are not limited to the above-mentioned problems, and other problems not mentioned will be clearly understood by those skilled in the art from the following description.

According to an aspect of the present disclosure, a pedicle screw assembly is provided, including a screw member extending in one direction and configured to be coupled to a pedicle; and a detachment-preventing member configured to rotatably support the screw member, wherein the screw member includes a screw body extending in the one direction, the screw body being configured to be at least partially inserted into the pedicle; a screw head positioned at one end of the screw body in the extending direction, the screw head being configured to be rotatably coupled to the detachment-preventing member about the one direction as an axis; and a screw protrusion coupled to the screw head opposite to the screw body with the screw head interposed therebetween, the screw protrusion being configured to be rotatably coupled to the detachment-preventing member about the one direction as an axis, wherein the detachment-preventing member includes detachment-preventing body including a first portion rotatably receiving the screw head and a second portion communicating with the first portion and rotatably receiving the screw protrusion, the second portion having a cross-sectional area smaller than that of the one portion.

In this case, a pedicle screw assembly may be provided, wherein the detachment-preventing body includes a detachment-preventing hollow formed through its interior along the one direction, the detachment-preventing hollow being configured to rotatably receive the screw head and the screw protrusion; a screw head support surface configured to rotatably support the screw head at one side of the one direction; and a screw protrusion support surface continuous with the screw head support surface, the screw protrusion support surface being configured to rotatably receive the screw protrusion at an opposite side of the one direction.

In addition, a pedicle screw assembly may be provided, wherein the screw head has an outer circumferential surface formed to be at least partially curved, and the screw head support surface is formed as a curved surface extending roundly to be convex radially outward.

In this case, a pedicle screw assembly may be provided, wherein the screw protrusion is configured to have a cross-section with a diameter smaller than a maximum diameter of a cross-section of the screw head; and the screw protrusion support surface is formed as a curved surface surrounding an outer circumference of the screw protrusion.

In addition, a pedicle screw assembly may be provided, including a rod support member coupled to the detachment-preventing member along the one direction, and configured to allow an external rod to pass through in engagement along a transverse direction; and a rod support nut coupled to the rod support member along the one direction opposite to the detachment-preventing member with the rod interposed therebetween.

In this case, a pedicle screw assembly may be provided, wherein the rod support member includes a member-receiving space located inside the rod support member and configured to accommodate the detachment-preventing member; a rod-receiving space communicating with the member-receiving space and configured to accommodate the rod; and a nut-receiving space communicating with the rod-receiving space and disposed opposite to the member-receiving space with the rod-receiving space interposed therebetween.

In addition, a pedicle screw assembly may be provided, wherein the rod support member includes a rod support thread formed on an inner surface of an outer circumference partially surrounding the nut-receiving space, the rod support thread being screw-coupled with a thread formed on an outer circumference of the rod support nut.

In this case, a pedicle screw assembly may be provided, wherein the rod support member is coupled to the detachment-preventing member at one side along the one direction and coupled to the rod support nut at an opposite side along the one direction, and the rod is positioned between the detachment-preventing member and the rod support nut.

In addition, a pedicle screw assembly may be provided, wherein the rod support nut is positioned between a first position supporting the rod to bias the rod into abutment with the detachment-preventing member and a second position supporting the rod to keep the rod spaced apart from the detachment-preventing member.

In this case, a pedicle screw assembly may be provided, wherein the rod support member includes a rod-coupling opening formed through an outer surface radially surrounding both the rod-receiving space and the nut-receiving space, the rod-coupling opening providing an insertion passage for the rod.

In addition, a pedicle screw assembly may be provided, wherein the rod support member includes a ring-receiving space communicating with the member-receiving space and disposed opposite to the rod-receiving space with the member-receiving space interposed therebetween, and the detachment-preventing member includes a detachment-preventing ring coupled to the screw head opposite to the screw protrusion and accommodated in the ring-receiving space.

In this case, a pedicle screw assembly may be provided, wherein the detachment-preventing ring has a hollow formed therein and a detachment-preventing opening recessed on an outer circumference thereof, and an inner diameter of the detachment-preventing ring is formed to be less than a maximum diameter of a cross-section of the screw head.

In addition, a pedicle screw assembly may be provided, wherein the detachment-preventing ring is received in the ring-receiving space after being radially pressed inward so that the detachment-preventing opening is at least partially reduced, and the detachment-preventing ring received in the ring-receiving space is radially shape-deformed inward so that the detachment-preventing opening is at least partially enlarged.

According to the above configuration, the pedicle screw assembly according to the embodiment of the present disclosure can minimize the invasive site.

In addition, according to the above configuration, the pedicle screw assembly according to the embodiment of the present disclosure can easily adjust the angle while being coupled to the spine.

In addition, according to the above configuration, the pedicle screw assembly according to an embodiment of the present disclosure can change a coupling position of a configuration connecting a plurality of screws without changing a coupling length of the screws.

In addition, according to the above configuration, the pedicle screw assembly according to an embodiment of the present disclosure can easily adjust the angle without a separation process while the screw is coupled to the spine.

In addition, according to the above configuration, the pedicle screw assembly according to an embodiment of the present disclosure can stably maintain the coupling state between the screw coupled to the spine and another component.

It should be understood that the effects of the present disclosure are not limited to the above-described effects, and include all effects that can be inferred from the configuration of the invention described in the detailed description or the claims.

### BRIEF DESCRIPTION OF THE DRAWINGS

FIG. 1 is a perspective view illustrating a pedicle screw assembly according to an embodiment of the present disclosure.

FIG. 2 is an exploded perspective view illustrating the configuration of the pedicle screw assembly of FIG. 1.

FIG. 3 is a perspective view illustrating a screw member provided in the pedicle screw assembly of FIG. 1.

FIG. 4 is a plan view illustrating the screw member of FIG. 3.

FIG. 5 is a cross-sectional view A-A illustrating the screw member of FIG. 3.

FIG. 6 is an exploded perspective view illustrating a detachment-preventing member and a rod support member provided in the pedicle screw assembly of FIG. 1.

FIG. 7 is an exploded plan view illustrating the detachment-preventing member and the rod support member of FIG. 6.

FIG. 8 is a cross-sectional view B-B illustrating the detachment-preventing member and the rod support member of FIG. 6.

FIG. 9 is a cross-sectional view C-C showing the detachment-preventing member and the rod support member of FIG. 6.

FIG. 10 is a perspective view illustrating a driver support member provided in the pedicle screw assembly of FIG. 1.

FIG. 11 is a cross-sectional view D-D illustrating the driver support member of FIG. 10.

FIG. 12 is a side cross-sectional view illustrating a rotation process of a pedicle screw assembly according to an embodiment of the present disclosure.

### DETAILED DESCRIPTION

Hereinafter, embodiments of the present disclosure will be described in detail with reference to the accompanying drawings so that those of ordinary skill in the art may readily implement the present disclosure. The present disclosure may be embodied in various forms and is not limited to the embodiments set forth herein. For clarity, parts not related to the description are omitted from the drawings, and like reference numerals refer to like or similar elements throughout the specification.

The words and terms used in the present specification and claims should not be construed as being limited to their ordinary or dictionary meanings, but should be interpreted in accordance with the principle that an inventor may define terms and concepts to best describe the invention, and should be understood as having meanings and concepts consistent with the technical spirit of the present disclosure.

Therefore, the embodiments described in this specification and the configurations illustrated in the drawings correspond to a preferred embodiment of the present disclosure and do not represent all the technical ideas of the present disclosure; accordingly, various equivalents and modifications that can replace the corresponding configurations may exist as of the filing date of the present disclosure.

In the following description, to clearly explain the features of the present disclosure, descriptions of some components may be omitted.

As used in the following description, the term “communicating” means that one or more members are connected to allow fluid communication with each other. In an embodiment, the communicating may be formed by members such as conduits, pipes, or tubing. In the following description, “communicating” may be used to have the same meaning as one or more members being “fluidly connected” to each other.

As used in the following description, the term “fluid” refers to any form of matter that flows under external force and whose shape or volume can be deformed. In an embodiment, the fluid may be a liquid such as water or a gas such as air.

The terms “upper side”, “lower side”, “left side”, “right side”, “front side” and “rear side” as used in the following description will be understood with reference to the coordinate system shown throughout the accompanying drawings.

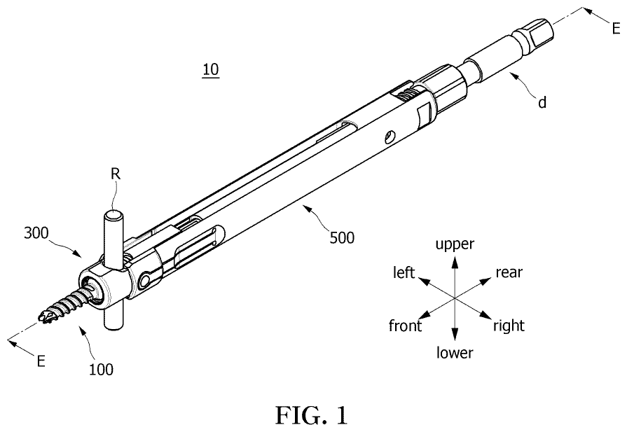

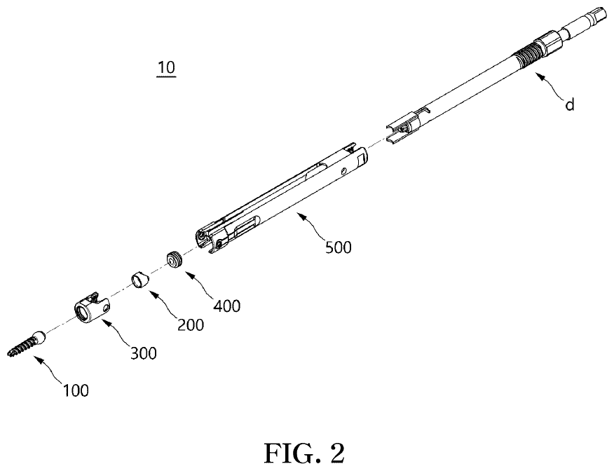

Referring to FIGS. 1 and 2, a pedicle screw assembly 10 according to an embodiment of the present disclosure is illustrated. The pedicle screw assembly 10 according to the embodiment of the present disclosure may be coupled to a pedicle P. A rod R that is separately provided is coupled to the pedicle screw assembly 10 coupled to the pedicle P so that an external force can be applied to the pedicle.

For this purpose, a plurality of pedicle screw assemblies 10 may be provided. The plurality of pedicle screw assemblies 10 may be coupled to pedicles at different positions. The rod is coupled to each of the plurality of pedicle screw assemblies 10 so that the external force can be applied to the pedicles through the rod.

In the following description, it is assumed that the pedicle screw assembly 10 is coupled to the pedicle of a human body. Alternatively, the pedicle screw assembly 10 according to an embodiment of the present disclosure may be provided as any device assuming that it is inserted into the human body and coupled to a bone.

The pedicle screw assembly 10 according to an embodiment of the present disclosure may be formed to have a length in one direction, that is, in a front-rear direction in the illustrated embodiment. The pedicle screw assembly 10 may be inserted into the human body along its longitudinal direction, and one side in the longitudinal direction, that is, a front side in the illustrated embodiment, may be coupled to the pedicle.

In addition, a configuration for forming a coupling with the pedicle may be coupled to the pedicle screw assembly 10 through the opposite side in its longitudinal direction.

At this time, the pedicle screw assembly according to an embodiment of the present disclosure includes a structure configured to be coupled to a pedicle, (that is, a screw member 100, and a structure configured to support the same (i.e., a detachment-preventing member 200 to be described later)) and the screw member is rotatable relative to the detachment-preventing member. Accordingly, the plurality of pedicle screw assemblies may be adjusted to appropriate angles for coupling with the rod while being in a state of being coupled to the spine, as some of the components are rotated.

At the same time, the structure configured to be coupled to the spine may have its movement in the longitudinal direction restricted by a structure configured to support the rod. Accordingly, the pedicle screw assembly may have only its angle adjusted while being maintained at a preset position so that the rod can be accurately coupled.

In the illustrated embodiment, the pedicle screw assembly includes a screw member, a detachment-preventing member, a rod support member, a rod support nut, and a driver support member.

In addition, as illustrated in FIGS. 1 and 2, the pedicle screw assembly 10 is coupled with a rod R and a driver d. In this case, the rod R and the driver d may be provided separately from the pedicle screw assembly 10.

The screw member 100 serves as a portion of the pedicle screw assembly 10 that is directly coupled to a pedicle. The screw member 100 may be fixedly coupled to the pedicle. In this case, the screw member 100 may be configured to be fixedly coupled to the pedicle by an external force, but may also be separable from the pedicle by the external force. In an embodiment, the screw member 100 may be provided in the form of a screw.

The screw member 100 constitutes one end in the longitudinal direction of the pedicle screw assembly 10. In the illustrated embodiment, the screw member 100 constitutes a front end of the pedicle screw assembly 10. The pedicle screw assembly 10 may be moved forward to be inserted into the human body and may be moved backward to be withdrawn from the human body.

The screw member 100 is coupled to a detachment-preventing member 200. Specifically, the screw member 100 may be coupled to the detachment-preventing member 200 so as to be rotatable about its longitudinal axis while being prevented from moving in the longitudinal direction. That is, the screw member 100 may be rotatably coupled to the detachment-preventing member 200.

As will be described later, the detachment-preventing member 200 may be rotatably coupled to the rod support member 300 about its longitudinal axis. Therefore, the screw member 100 may also be rotatably coupled to the rod support member 300.

The screw member 100 is coupled to the rod support member 300. The screw member 100 may be coupled to the rod support member 300 via the detachment-preventing member 200. The screw member 100 may be coupled to the rod support member 300 so as to be rotatable about its longitudinal axis while being prevented from moving in the longitudinal direction.

The driver d may be detachably coupled to the screw member 100. After the pedicle screw assembly 10 is inserted into a human body, the driver d may pass through the rod support member 300, the rod support nut 400, and the driver support member 500 and be coupled to one end of the screw member 100 in its longitudinal direction.

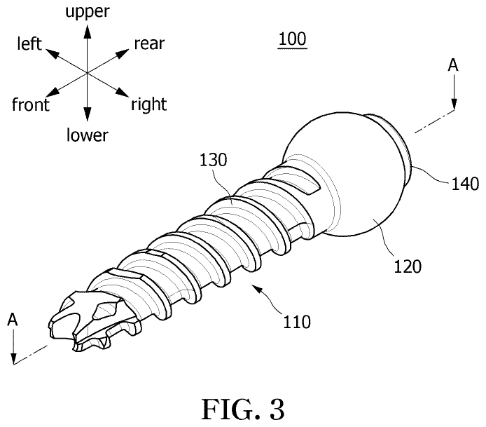

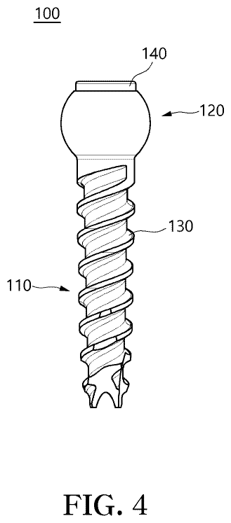

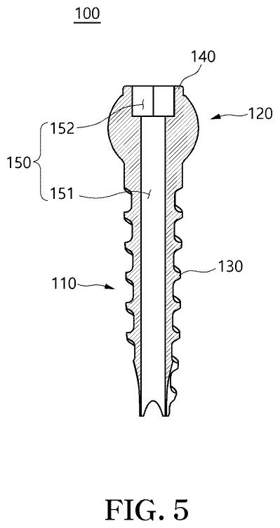

In the embodiment illustrated in FIGS. 3 to 5, screw member 100 includes a screw body 110, a screw head 120, a screw thread 130, a screw protrusion 140, and a screw hollow 150.

The screw body 110 constitutes a part of the outer shape of screw member 100. The crew body 110 extends in an extending direction of screw member 100, that is, in a front-rear direction in the illustrated embodiment.

The screw body 110 is coupled with or formed with other components of the screw member 100. In the illustrated embodiment, one end in the extending direction of the screw body 110, that is, a rear end, is coupled to the screw head 120. The screw thread 130 is formed on an outer circumferential surface of the screw body 110, and the screw hollow 150 is formed inside the screw body 110.

The screw body 110 is a portion of the pedicle screw assembly 10 that is directly coupled to the spine. To this end, the screw body 110 may have a shape that allows easy insertion into the pedicle. In the illustrated embodiment, the screw body 110 is formed in a cone or frusto-conical shape in which a cross-sectional area increases from an opposite end in the extending direction, that is, a front end, toward one end in the extending direction, that is, a rear end.

The screw head 120 constitutes another portion of an outer shape of the screw member 100. The screw head 120 is coupled to one end of the screw body 110 in the extending direction, that is, a rear end in the illustrated embodiment.

The screw head 120 is coupled to the detachment-preventing member 200. Specifically, the screw head 120 is rotatably accommodated in a detachment-preventing hollow 211 of a detachment-preventing body 210. The screw head 120 is rotatably supported by a screw head support surface 212 that surrounds the detachment-preventing hollow 211.

Accordingly, it will be understood that the screw member 100 is rotatably coupled to the detachment-preventing member 200.

The screw head 120 may have any shape that can be rotatably coupled with the detachment-preventing member 200. In the illustrated embodiment, the screw head 120 is configured as a portion of a sphere, that is, its outer circumferential surface is formed as a curved surface.

One side of the screw head 120 in a height direction, that is, a lower side in the illustrated embodiment, is continuous with an upper end of the screw body 110. An opposite side of the screw head 120 in the height direction, that is, an upper side in the illustrated embodiment, is provided with the screw protrusion 140.

As will be described later, the screw protrusion 140 is supported by a screw protrusion support surface 213 and a rod R, and its movement in an axial direction, that is, a front-rear direction in the illustrated embodiment, is restricted. Accordingly, it will be understood that the screw head 120 is capable of only rotational motion about a center axis of the screw body 110.

A screw thread 130 is formed on an outer circumferential surface of the screw body 110. The screw thread 130 is configured to maintain a coupling state between the screw body 110 and the spine. The screw thread 130 is coupled to an inner surface of the spine.

The screw thread 130 extends obliquely along a length direction of the screw body 110. At this time, the screw thread 130 may extend in a helical shape. The screw thread 130 protrudes outward from the outer surface of the screw body 110.

The screw protrusion 140 constitutes a portion of the screw member 100 that is supported by the detachment-preventing member 200 and the rod R. The screw protrusion 140 is rotatably coupled to the detachment-preventing member 200 together with the screw head 120. At this time, the screw protrusion 140 is restricted in movement in a length direction of the screw member 100, that is, a front-rear direction, by a screw protrusion support surface 213 of the detachment-preventing member 200 and the rod R.

Accordingly, with the screw protrusion 140 provided, the pedicle screw assembly 10 according to an embodiment of the present disclosure may be rotated about the longitudinal axis of the screw member 100, while movement in the length direction may be restricted.

The screw protrusion 140 is continuous with the screw head 120. In the illustrated embodiment, the screw protrusion 140 is located at the opposite side in a height direction of the screw head 120, that is, a rear side. The screw protrusion 140 is disposed opposite to the screw body 110 and the screw thread 130 with the screw head 120 interposed therebetween.

The screw protrusion 140 may have any shape that can come into contact with the detachment-preventing body 210 or the rod R to restrict movement of the screw member 100 in a longitudinal direction. In the illustrated embodiment, the screw protrusion 140 is formed in a disk shape having a circular cross-section and a thickness in a front-rear direction.

At this time, a diameter of a cross-section of the screw protrusion 140 may be formed to be less than a maximum value of a diameter of a cross-section of the screw head 120. As will be described later, the shortest distance between screw head support surfaces 212 is formed to be greater than the longest distance between screw protrusion support surfaces 213.

Accordingly, the screw head 120 is supported by the screw head support surface 212, so that it is prevented from moving in the longitudinal direction.

A screw hollow 150 is a space formed inside the screw member 100. The screw hollow 150 provides a space into which a driver d is inserted. In addition, the screw hollow 150 reduces an overall weight of the screw member 100 and allows the screw member 100 to be easily coupled to the spine.

The screw hollow 150 is formed to penetrate longitudinally inside the screw member 100. In the illustrated embodiment, the screw hollow 150 is formed to penetrate through the screw body 110, the screw head 120, and the screw protrusion 140 along the front-rear direction.

The screw hollow 150 may be divided into a plurality of portions. The plurality of portions communicates with each other, but may have different cross-sectional areas. In the illustrated embodiment, the screw hollow 150 includes a first screw hollow 151 and a second screw hollow 152.

The first screw hollow 151 is a portion of the screw hollow 150 that is partially formed in the screw body 110 and the screw head 120. Each end of the first screw hollow 151 in the longitudinal direction, that is, a front end and a rear end in the illustrated embodiment, is open.

The second screw hollow 152 is a portion of the screw hollow 150 that is formed in a remaining portion of the screw head 120 and in the screw protrusion 140. Each end of the second screw hollow 152 in the longitudinal direction, that is, a front end and a rear end in the illustrated embodiment, is open. The driver d may be inserted into the second screw hollow 152.

At this time, a diameter of a cross-section of the first screw hollow 151 may be formed to be equal to or less than a diameter of a cross-section of the second screw hollow 152. Preferably, the diameter of the cross-section of the first screw hollow 151 may be formed to be less than the diameter of the cross-section of the second screw hollow 152 or a diameter of the driver d.

Accordingly, the driver d may be inserted into the second screw hollow 152 to apply a rotational force to the screw member 100, but insertion of the driver d into the first screw hollow 151 may be restricted.

The detachment-preventing member 200 maintains a coupling state between the screw member 100 and a rod support member 300. The detachment-preventing member 200 is respectively coupled to the screw member 100 and the rod support member 300.

Specifically, the detachment-preventing member 200 surrounds and is coupled to an opposite side in a longitudinal direction of the screw member 100, that is, a rear side in the illustrated embodiment. The opposite side in the longitudinal direction of the screw member 100 may be accommodated inside the detachment-preventing member 200. At this time, the detachment-preventing member 200 may rotatably accommodate the opposite side in the longitudinal direction of the screw member 100.

The detachment-preventing member 200 is coupled to the rod support member 300. The detachment-preventing member 200 is accommodated in a member-receiving space 310 formed inside the rod support member 300. The detachment-preventing member 200 may be disposed so as not to be exposed to an outside of the rod support member 300.

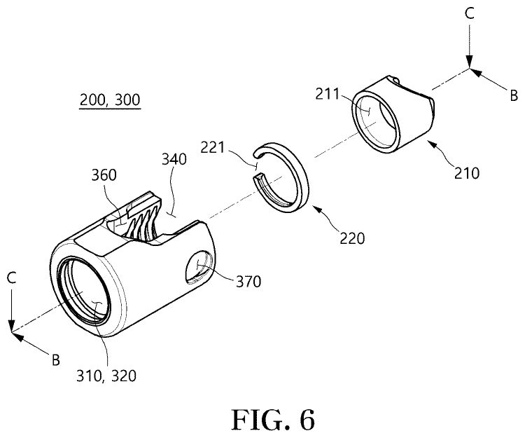

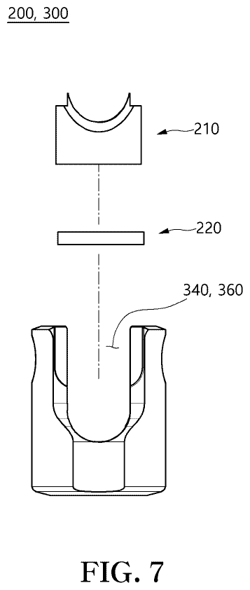

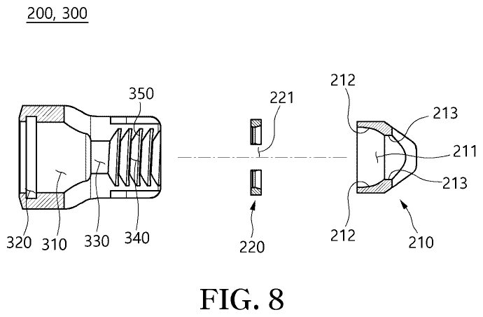

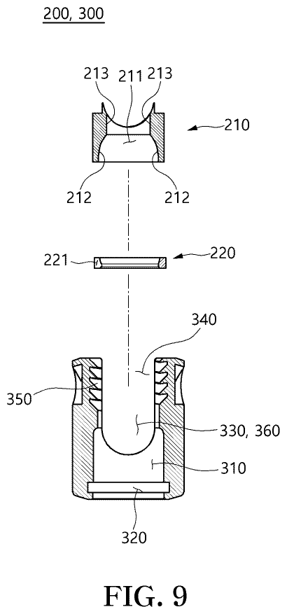

In the embodiments illustrated in FIGS. 6 to 9, the detachment-preventing member 200 includes a detachment-preventing body 210 and a detachment-preventing ring 220.

The detachment-preventing body 210 rotatably accommodates a portion of the opposite side in the longitudinal direction of the screw member 100, specifically, the screw head 120 and the screw protrusion 140. The detachment-preventing body 210 is accommodated in a member-receiving space 310 of the rod support member 300 and is supported by an inner surface of the rod support member 300. In an embodiment, the detachment-preventing body 210 may rotatably accommodate the screw member 100 and may be rotatably accommodated in the member-receiving space 310.

At the same time, the detachment-preventing body 210 may restrict movement of the screw head 120 and the screw protrusion 140 in a longitudinal direction, that is, a front-rear direction in the illustrated embodiment. Accordingly, the screw member 100 coupled to the detachment-preventing body 210 may be rotated about its longitudinal direction as an axis, while movement in the longitudinal direction may be restricted.

In the illustrated embodiment, the detachment-preventing body 210 includes a detachment-preventing hollow 211, a screw head support surface 212, and a screw protrusion support surface 213.

The detachment-preventing hollow 211 rotatably accommodates the screw head 120 and the screw protrusion 140 continuous with the screw head 120. The detachment-preventing hollow 211 is defined as a space formed inside the detachment-preventing body 210.

The detachment-preventing hollow 211 may have a shape corresponding to a shape of the detachment-preventing body 210. In the illustrated embodiment, a three-dimensional space having partially different shapes along a height direction, which has a circular cross-section and a height in a front-rear direction, is formed as the detachment-preventing hollow 211.

Each end of the detachment-preventing hollow 211 in a longitudinal direction, that is, a front side and a rear side in the illustrated embodiment, is open. The screw head 120 and the screw protrusion 140 may be accommodated in the detachment-preventing hollow 211 through the front end. The driver d may access the screw head 120 through the rear end.

The detachment-preventing hollow 211 may include a first portion that is at least partially spherical. In the first portion, a screw head 120 formed in a spherical shape is rotatably accommodated. The first portion is defined by being surrounded by the screw head support surface 212.

The detachment-preventing hollow 211 may include a second portion that is at least partially disk-shaped. In the second portion, the screw protrusion 140 formed in a disk shape is rotatably accommodated. The second portion is defined by being surrounded by the screw protrusion support surface 213.

The first portion and the second portion communicate with each other. Accordingly, the screw head 120 and the screw protrusion 140, which are continuously coupled to each other, can be accommodated together in the detachment-preventing hollow 211. In the illustrated embodiment, the first portion is located on a front side, and the second portion is located on a rear side.

At this time, a diameter of a cross-section of the second portion of the detachment-preventing hollow 211 may be formed to be equal to or less than a minimum value of a diameter of a cross-section of the first portion. Accordingly, the screw head 120 accommodated in the portion is prevented from entering the second portion.

The screw head support surface 212 surrounds the first portion of the detachment-preventing hollow 211 in a radial direction. The screw head support surface 212 rotatably supports the screw head 120 accommodated in the detachment-preventing hollow 211 in the radial direction.

The screw head support surface 212 is defined as a portion of an inner surface of the detachment-preventing body 210. In the illustrated embodiment, the screw head support surface 212 is defined as a lower portion of the inner surface of the detachment-preventing body 210.

The screw head support surface 212 may have a shape corresponding to an outer circumferential surface of the screw head 120. In the illustrated embodiment, the screw head support surface 212 extends along an inner circumference of the detachment-preventing body 210 and is formed as a rounded curved surface that is convex radially outward.

The screw head support surface 212 surrounds the first portion of the detachment-preventing hollow 211 in a radial direction. At this time, along the diameter direction of the detachment-preventing hollow 211, the minimum distance between the screw head support surfaces 212 may be greater than the maximum distance between the screw protrusion support surfaces 213 along the same direction.

By the above-described structure, the screw head 120 accommodated in the first portion of the detachment-preventing hollow 211 is prevented from moving into the second portion of the detachment-preventing hollow 211.

The screw protrusion support surface 213 surrounds the second portion of the detachment-preventing hollow 211 in a radial direction. The screw protrusion support surface 213 rotatably supports the screw protrusion 140 accommodated in the detachment-preventing hollow 211 in the radial direction.

The screw protrusion support surface 213 is defined as second portion of an inner surface of the detachment-preventing body 210. In the illustrated embodiment, the screw protrusion support surface 213 is defined as an intermediate portion or an upper portion of the inner surface of the detachment-preventing body 210. The screw protrusion support surface 213 is continuous with the screw head support surface 212.

The screw protrusion support surface 213 may have a shape corresponding to an outer circumferential surface of the screw protrusion 140. In the illustrated embodiment, the screw protrusion support surface 213 is formed as a curved surface extending along an inner circumference of the detachment-preventing body 210.

The screw protrusion support surface 213 extends while surrounding the second portion of the detachment-preventing hollow 211. As described above, the maximum distance between the screw protrusion support surfaces 213 along the radial direction of the detachment-preventing hollow 211 may be less than the minimum distance between the screw head support surfaces 212 along the same direction.

The detachment-preventing ring 220 rotatably accommodates another portion of the opposite side of the screw member 100 in a longitudinal direction. The detachment-preventing ring 220 is formed to have an outer diameter greater than that of the detachment-preventing body 210.

The detachment-preventing ring 220 prevents the detachment-preventing body 210 accommodated in the member-receiving space 310 from being detached in an axial direction of the screw member 100, that is, toward a front side in the illustrated embodiment. The detachment-preventing ring 220 supports the detachment-preventing body 210 at one side in a longitudinal direction, that is, on the front side.

The detachment-preventing ring 220 is coupled to the screw member 100. The detachment-preventing ring 220 is positioned closer to one side in a longitudinal direction of the screw member 100 than the detachment-preventing body 210, that is, toward the front side in the illustrated embodiment. The detachment-preventing ring 220 is located between a front end of the rod support member 300 and the detachment-preventing body 210.

The detachment-preventing ring 220 is formed in an annular shape and may have a detachment-preventing opening 221 formed on one side. The detachment-preventing opening 221 may constitute a space allowing the detachment-preventing ring 220 to be deformed in shape so that its outer diameter is reduced when pressed. The detachment-preventing ring 220 may be coupled to the rod support member 300 after being radially pressed inward and deformed in shape in a state in which the screw member 100 passes through the detachment-preventing ring 220.

When the pressing state is released, the detachment-preventing ring 220 may be elastically deformed radially outward and may come into close contact with the rod support member 300. Accordingly, the detachment-preventing ring 220 can stably support the detachment-preventing body 210 and a rod R positioned at its rear side.

At this time, an inner diameter of the detachment-preventing ring 220 may be formed to be less than a minimum value of a diameter of a cross-section of the screw head 120. Accordingly, the detachment-preventing ring 220 can prevent the screw head 120 accommodated in the detachment-preventing hollow 211 from being arbitrarily withdrawn.

Accordingly, it will be understood that a rearward movement of the screw member 100 coupled to the detachment-preventing member 200 is prevented by the screw protrusion support surface 213 and the rod R coupled thereto, and a frontward movement of the screw member 100 is prevented by the detachment-preventing ring 220.

Accordingly, the screw member 100 is rotatably coupled to the detachment-preventing member 200, while its movement in its longitudinal direction may be restricted.

The rod support member 300 is a component to which an external rod R is coupled. The rod support member 300 supports the external rod R. The external rod R may extend along the pedicle or the spine by a plurality of rod support members 300 respectively provided in a plurality of pedicle screw assemblies 10.

The rod support member 300 is coupled to the screw member 100. Specifically, the rod support member 300 is coupled to the screw member 100 through the detachment-preventing member 200. The rod support member 300 may rotatably accommodate the detachment-preventing member 200 that rotatably supports the screw member 100.

The rod support member 300 extends in a longitudinal direction of the screw member 100, that is, in a front-rear direction in the illustrated embodiment. One side in an extending direction of the rod support member 300, that is, a front side in the illustrated embodiment, surrounds the screw member 100 and the detachment-preventing member 200 coupled thereto. The opposite in the extending direction of the rod support member 300, that is, a rear side in the illustrated embodiment, is coupled to a driver support member 500.

A hollow is formed inside the rod support member 300. The driver d for operating the screw member 100 may be insertable into and withdrawable from the rod support member 300.

The rod support member 300 is coupled to a driver support member 500. The rod support member 300 may be at least partially accommodated in the driver support member 500. A portion of the rod support member 300 may be withdrawn together with the driver support member 500 to an outside of the human body.

In the embodiments illustrated in FIGS. 6 to 9, the rod support member 300 includes a member-receiving space 310, a ring-receiving space 320, a rod-receiving space 330, a nut-receiving space 340, a rod support thread 350, a rod-coupling opening 360, and a protrusion-coupling groove 370.

The member-receiving space 310 is a space that accommodates a detachment-preventing body 210 of the detachment-preventing member 200. The member-receiving space 310 is defined as a portion of a space formed inside the rod support member 300. In the illustrated embodiment, the member-receiving space 310 is located at a rear side of a ring-receiving space 320 along a longitudinal direction, that is, along a front-rear direction of the rod support member 300.

The member-receiving space 310 may have a shape corresponding to a shape of the detachment-preventing body 210. In the illustrated embodiment, the member-receiving space 310 has partially different shapes along a height direction, which has a circular cross-section and a height in a front-rear direction. In the illustrated embodiment, a front side of the member-receiving space 310 is cylindrical. In addition, a rear side of the member-receiving space 310 is formed in a frusto-conical shape in which a cross-sectional area decreases toward the rear side.

The member-receiving space 310 communicates with a ring-receiving space 320 and a rod-receiving space 330. One side of the member-receiving space 310 in a height direction, that is, a front side in the illustrated embodiment, communicates with the ring-receiving space 320. The opposite side of the member-receiving space 310 in the height direction, that is, a rear side in the illustrated embodiment, communicates with the rod-receiving space 330.

The ring-receiving space 320 is a space that accommodates a detachment-preventing ring 220 of the detachment-preventing member 200. The detachment-preventing ring 220 is accommodated in the ring-receiving space 320 in a state in which it is pressed so that the detachment-preventing opening 221 is closed, and then restored to its original shape, thereby allowing the detachment-preventing opening 221 to be formed again.

The ring-receiving space 320 is defined as another portion of a space formed inside the rod support member 300. In the illustrated embodiment, the ring-receiving space 320 is located on a front side of the member-receiving space 310 along a longitudinal direction, that is, along a front-rear direction of the rod support member 300. The ring-receiving space 320 is disposed opposite to the rod-receiving space 330 with the member-receiving space 310 interposed therebetween.

The ring-receiving space 320 may have a shape corresponding to a shape of the detachment-preventing ring 220. In the illustrated embodiment, the ring-receiving space 320 is formed as a disk-shaped space having a circular cross-section and a height in a front-rear direction.

The ring-receiving space 320 communicates with the member-receiving space 310. At this time, an outer diameter of a cross-section of the ring-receiving space 320 may be formed to exceed a maximum value of an outer diameter of a cross-section of the member-receiving space 310. Accordingly, the detachment-preventing ring 220 accommodated in the ring-receiving space 320 is prevented from being arbitrarily detached from the ring-receiving space 320 and moving into the member-receiving space 310.

The rod-receiving space 330 movably accommodates the rod R. The rod-receiving space 330 is defined as another portion of a space formed inside the rod support member 300.

In the illustrated embodiment, the rod-receiving space 330 is located at a rear side of the member-receiving space 310 along a longitudinal direction, that is, along a front-rear direction of the rod support member 300. The rod-receiving space 330 is disposed opposite to the ring-receiving space 320 with the member-receiving space 310 interposed therebetween.

The rod-receiving space 330 may have a shape corresponding to a shape of the rod R. As illustrated in FIG. 1, the rod R has a cylindrical shape having a circular cross-section and a height in one direction, and the rod-receiving space 330 may be formed as a space having a width and a height equal to or greater than a cross-sectional area of the rod R.

The rod-receiving space 330 communicates with the member-receiving space 310. The rod R accommodated in the rod-receiving space 330 may support the detachment-preventing body 210 accommodated in the member-receiving space 310.

The rod-receiving space 330 communicates with a rod-coupling opening 360. The rod R may pass through the rod-receiving space 330 via the rod-coupling opening 360 and extend outward of the rod support member 300.

The nut-receiving space 340 is a space that accommodates a rod support nut 400. The nut-receiving space 340 is defined as another portion of a space formed inside the rod support member 300. In the illustrated embodiment, the nut-receiving space 340 is located at a rearmost side of the rod support member 300 along a longitudinal direction, that is, along a front-rear direction. The nut-receiving space 340 is disposed opposite to the member-receiving space 310 with the rod-receiving space 330 interposed therebetween.

The nut-receiving space 340 may have a shape corresponding to a shape of the rod support nut 400. In the illustrated embodiment, the nut-receiving space 340 is formed as a cylindrical space having a circular cross-section and a height in a front-rear direction.

The nut-receiving space 340 communicates with the rod-receiving space 330. The rod support nut 400 accommodated in the nut-receiving space 340 may press the rod R accommodated in the rod-receiving space 330 toward one side in a longitudinal direction, that is, toward a front side in the illustrated embodiment. Accordingly, it can be understood that the rod support nut 400 supports both the rod R and the detachment-preventing member 200.

The nut-receiving space 340 communicates with the rod-coupling opening 360. Each side of the nut-receiving space 340 in a height direction, that is, an upper side and a lower side in the illustrated embodiment, communicates with an exterior through the rod-coupling opening 360.

Accordingly, the rod R may enter the rod-receiving space 330 or the nut-receiving space 340 through the rod-coupling opening 360, and then be moved toward the member-receiving space 310 by the rod support nut 400 to support the detachment-preventing member 200 and the screw member 100 rotatably coupled thereto.

A rod support thread 350 is formed on an inner circumferential surface of the rod support member 300 surrounding the nut-receiving space 340.

The rod support thread 350 is coupled to the rod support nut 400. The rod support thread 350 is formed on the inner circumferential surface of the rod support member 300 surrounding the nut-receiving space 340. The rod support thread 350 may be screw-coupled with a thread formed on an outer circumference of the rod support nut 400.

As the rod support thread 350 is formed, a distance by which the rod support nut 400 is accommodated in the nut-receiving space 340 can be adjusted. As a result, a position of the rod R supported by the rod support nut 400 can also be adjusted.

The rod-coupling opening 360 is defined as an opening formed through an outer surface of the rod support member 300. The rod-coupling opening 360 communicates the rod-receiving space 330 and the nut-receiving space 340 with an exterior. The rod-coupling opening 360 respectively communicates with the rod-receiving space 330 and the nut-receiving space 340.

Accordingly, as described above, the rod R may be partially accommodated in the rod-receiving space 330 or the nut-receiving space 340 through the rod-coupling opening 360, and then moved toward the detachment-preventing member 200 by the rod support nut 400.

The rod-coupling opening 360 is formed through a surface of the rod support member 300 in a height direction. The rod-coupling opening 360 extends in a longitudinal direction of the rod support member 300, that is, in a front-rear direction in the illustrated embodiment. One side of the rod-coupling opening 360 in the longitudinal direction, that is, a front side in the illustrated embodiment, is closed. The rod R may be supported by the closed front side of the rod-coupling opening 360.

The opposite side of the rod-coupling opening 360 in a longitudinal direction, that is, a rear side in the illustrated embodiment, is open. In an embodiment, the rod R may also enter the rod-receiving space 330 or the nut-receiving space 340 through the opposite side, that is, the rear side of the rod-coupling opening 360.

A plurality of rod-coupling openings 360 may be formed. In the illustrated embodiment, the rod-coupling openings 360 are respectively formed on upper and lower surfaces of the rod support member 300. The pair of rod-coupling openings 360 are disposed opposite to each other with the rod-receiving space 330 or the nut-receiving space 340 interposed therebetween.

The protrusion-coupling groove 370 is a portion where the rod support member 300 is coupled to the driver support member 500. The protrusion-coupling groove 370 accommodates a support-member coupling protrusion 530 provided on the driver support member 500.

As will be described later, the support-member coupling protrusion 530 is formed to be inclined such that its cross-sectional area increases in a direction opposite to the detachment-preventing member 200. Accordingly, the support-member coupling protrusion 530 accommodated in the protrusion-coupling groove 370 may not be separated arbitrarily without application of an external force.

The protrusion-coupling groove 370 may have a shape corresponding to a shape of the support-member coupling protrusion 530. In the illustrated embodiment, the protrusion-coupling groove 370 is formed as a disk-shaped space having a circular cross-section and a thickness in a left-right direction.

The protrusion-coupling groove 370 may be located in a portion of the rod support member 300 where the rod-coupling opening 360 is not formed. In the illustrated embodiment, the protrusion-coupling groove 370 is formed through a surface of the rod support member 300 in a width direction.

A plurality of protrusion-coupling grooves 370 may be formed. The plurality of protrusion-coupling grooves 370 may be spaced apart from each other and formed at different positions. In the illustrated embodiment, a pair of protrusion-coupling grooves 370 are formed. The pair of protrusion-coupling grooves 370 are respectively formed through left and right surfaces, that is, opposite sides in a width direction of the rod support member 300.

Accordingly, it can be understood that the rod-coupling openings 360 and the protrusion-coupling grooves 370 are alternately arranged along an outer circumference of the rod support member 300.

Referring again to FIG. 1, a pedicle screw assembly 10 according to an embodiment of the present disclosure includes a rod support nut 400.

The rod support nut 400 is coupled to the rod support member 300 and supports the rod R. The rod support nut 400 presses the rod R toward one side in a longitudinal direction of the rod support member 300, that is, toward a front side in the illustrated embodiment. In other words, the rod support nut 400 supports the rod R from the opposite to side in the longitudinal direction of the rod support member 300, that is, from a rear side in the illustrated embodiment.

A hollow is formed through an interior of the rod support nut 400. The hollow communicates with the member-receiving space 310, the ring-receiving space 320, the rod-receiving space 330, and the nut-receiving space 340 formed inside the rod support member 300. In addition, the hollow communicates with a driver-receiving space 510 and a support-member receiving space 520 to form a passage for penetration of the driver d.

The rod support nut 400 is coupled to the rod support member 300. The rod support nut 400 is accommodated in the nut-receiving space 340 and screw-coupled with the rod support thread 350. The rod support nut 400 is disposed opposite to the detachment-preventing member 200 with the rod R interposed therebetween.

Meanwhile, a distance by which the rod support nut 400 is coupled to the rod support member 300 can be adjusted. In other words, a ratio of a portion of the rod support nut 400 accommodated in the nut-receiving space 340 can be changed.

Accordingly, the rod support nut 400 may be accommodated in the nut-receiving space 340 until the rod R comes into contact with the detachment-preventing member 200, or may be accommodated in the nut-receiving space 340 such that the rod R is spaced apart from the detachment-preventing member 200.

Accordingly, even when a length by which the screw member 100 is coupled to the spine needs to be changed, a coupling height of the rod R can be adjusted by changing a position of the rod support nut 400 without adjusting a coupling length of the screw member 100 itself.

The driver support member 500 accommodates and supports the driver d. The driver d is prevented from oscillating in any direction, that is, in the radial direction, by the driver support member 500, and can be moved along the longitudinal direction to be coupled to the screw member 100.

The driver support member 500 is coupled to the rod support member 300. The driver support member 500 can accommodate the rod support member 300 at least partially. In an embodiment, the driver support member 500 may be hook-coupled to the rod support member 300.

The driver support member 500 extends in a direction corresponding to an insertion and withdrawal direction of the pedicle screw assembly 10. In the illustrated embodiment, the driver support member 500 extends in a front-rear direction. One side of the driver support member 500 in a longitudinal direction, that is, a front side in the illustrated embodiment, is coupled to the rod support member 300.

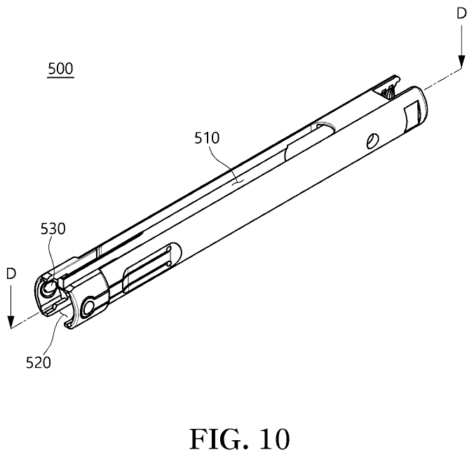

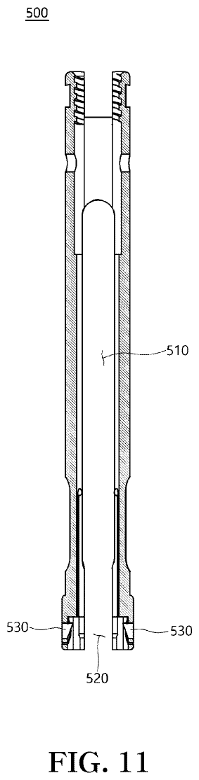

In the embodiments illustrated in FIGS. 10 and 11, the driver support member 500 includes a driver-receiving space 510, a support-member receiving space 520, and a support-member coupling protrusion 530.

The driver-receiving space 510 is a space formed inside the driver support member 500. The driver-receiving space 510 accommodates the driver d. The driver-receiving space 510 extends in a longitudinal direction of the driver support member 500, that is, in a front-rear direction in the illustrated embodiment. Each side of the driver-receiving space 510 in the longitudinal direction, that is, a front side and a rear side in the illustrated embodiment, is open to form a passage through which the driver d can pass.

The driver-receiving space 510 may have a shape corresponding to a shape of the driver d. In the illustrated embodiment, the driver-receiving space 510 is formed as a columnar space having a predetermined cross-section and a length in a front-rear direction.

A space located on one side of the driver-receiving space 510 in a longitudinal direction, that is, on a front side in the illustrated embodiment, may be defined as a support-member receiving space 520.

The support-member receiving space 520 is a space that accommodates the rod support member 300. A portion of the rod support member 300 in its longitudinal direction may be accommodated in the support-member receiving space 520. In an embodiment, the rod support member 300 may be inserted and accommodated in the support-member receiving space 520 until the support-member coupling protrusion 530 is inserted into the protrusion-coupling groove 370.

The support-member receiving space 520 may have a shape corresponding to a shape of the rod support member 300. In the illustrated embodiment, the support-member receiving space 520 is formed as a space having a cross-section partially including a curved line and extending in a front-rear direction.

A support-member coupling protrusion 530 is positioned in the support-member receiving space 520.

The support-member coupling protrusion 530 is a portion where the driver support member 500 is coupled to the rod support member 300. The support-member coupling protrusion 530 is inserted into the protrusion-coupling groove 370. The inserted support-member coupling protrusion 530 is not arbitrarily withdrawn from the protrusion-coupling groove 370 without application of an external force.

The support-member coupling protrusion 530 is located on an inner surface of the driver support member 500 surrounding the support-member receiving space 520. In the illustrated embodiment, the support-member coupling protrusion 530 protrudes from an inner surface of the driver support member 500 in a width direction.

The support-member coupling protrusion 530 may have any shape that can be easily inserted into the protrusion-coupling groove 370 but not arbitrarily withdrawn therefrom. In the embodiment illustrated in FIG. 11, the support-member coupling protrusion 530 is formed such that its cross-sectional area increases in a direction from one side to the opposite side in a longitudinal direction of the driver support member 500, that is, from a front side toward a rear side in the illustrated embodiment.

In addition, the support-member coupling protrusion 530 extends obliquely inward of the driver support member 500 in a direction from the one side toward the opposite side, that is, from the front side toward the rear side in the illustrated embodiment. That is, the support-member coupling protrusion 530 is formed such that a length of a portion protruding at the rear side is greater than a length of a portion protruding at the front side.

Accordingly, when the rod support member 300 is moved toward the driver support member 500, the rod support member 300 comes into contact with an inclined surface of the support-member coupling protrusion 530 and can press the driver support member 500 outward. When the support-member coupling protrusion 530 is accommodated in the protrusion-coupling groove 370, the support-member coupling protrusion 530 is prevented from being arbitrarily withdrawn from the protrusion-coupling groove 370 by a rear surface of the support-member coupling protrusion 530, that is, a surface extending in a left-right direction.

A plurality of support-member coupling protrusions 530 may be provided. The plurality of support-member coupling protrusions 530 may be spaced apart from each other and disposed at different positions. In the illustrated embodiment, a pair of support-member coupling protrusions 530 are provided and spaced apart in a width direction, that is, in a left-right direction of the driver support member 500. The pair of support-member coupling protrusions 530 are respectively located on left and right inner surfaces of the driver support member 500. The pair of support-member coupling protrusions 530 are disposed opposite to each other with the support-member receiving space 520 interposed therebetween.

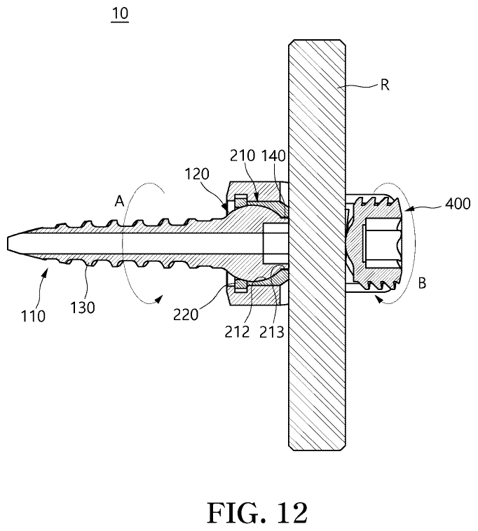

Referring to FIG. 12, coupling structures of respective components of a pedicle screw assembly 10 according to an embodiment of the present disclosure are illustrated as an example.

In the illustrated embodiment, the screw member 100 is rotatably coupled to the detachment-preventing member 200. In addition, the detachment-preventing member 200 is coupled to the rod support member 300. The rod R also passes through the rod-coupling opening 360 and is coupled to the rod support member 300.

At this time, the screw head 120 is accommodated in the detachment-preventing hollow 211 and is rotatably supported by a screw head support surface 212 configured to include a curved surface rounded to be convex radially outward. In addition, the screw protrusion 140 continuous with the screw head 120 is also accommodated in the detachment-preventing hollow 211 and is rotatably supported by a screw protrusion support surface 213 formed in a shape corresponding to an inner circumference of a disk.

In this state, when the screw body 110 is coupled to the spine, the screw member 100 itself is fixed. Accordingly, relative rotation between the screw member 100 and the detachment-preventing member 200 can be achieved as the detachment-preventing member 200 is rotated counterclockwise (A) or clockwise (B) about the longitudinal direction of the screw member 100 as an axis.

Meanwhile, as described above, the member-receiving space 310 that accommodates the detachment-preventing member 200 communicates with the rod-receiving space 330 that accommodates the rod R. In addition, since each end of the detachment-preventing hollow 211 in the longitudinal direction is open, the detachment-preventing hollow 211 communicates with the rod-receiving space 330.

Accordingly, the screw protrusion 140 accommodated in the detachment-preventing hollow 211 may be supported by the rod R accommodated in the rod-receiving space 330. Meanwhile, the rod support nut 400 is positioned in the nut-receiving space 340 communicating with the rod-receiving space 330. The rod support nut 400 can support the rod R.

Accordingly, movement of the screw member 100 in a direction in which the screw member 100 is separated from the coupled spine, that is, in a direction toward the rod R, can be restricted.

Furthermore, a detachment-preventing ring 220 is coupled to one portion of the screw head 120, that is, the front portion in the illustrated embodiment of FIG. 12. The inner diameter of a cross-section of the detachment-preventing ring 220, that is, the diameter of the hollow formed inside the detachment-preventing ring 220, is formed to be less than the diameter of the cross-section of the one portion of the screw head 120.

Accordingly, movement of the screw member 100 in a direction toward the coupled spine, that is, in a direction opposite to the rod R, can also be restricted.

As a result, in the pedicle screw assembly 10 according to an embodiment of the present disclosure, while the screw member 100 is rotatably coupled with other components, its movement in an axial direction can be prevented by the screw protrusion 140.

Meanwhile, in the illustrated embodiment, the rod support nut 400 is coupled until the rod R comes into contact with the detachment-preventing body 210 and the screw protrusion 140 coupled thereto. As described above, the coupling distance of the rod support nut 400 can be adjusted. When the coupling position of the rod R needs to be adjusted further rearward, the rod support nut 400 may be received in the nut-receiving space 340 only to a distance where the rod R can be spaced apart from the detachment-preventing body 210 or the screw protrusion 140.

That is, by changing the position of the rod support nut 400, the coupling position of the rod R can be adjusted without changing the insertion length of the screw body 110. Accordingly, insertion and withdrawal of the screw member 100 coupled to the spine are not required, thereby reducing pain of the patient, improving surgical efficiency, and minimizing invasiveness.

Although exemplary embodiments of the present disclosure have been described above, the scope of the present disclosure is not limited to the embodiments set forth herein. Those skilled in the art who understand the spirit of the present disclosure may readily propose other embodiments by adding, modifying, deleting, or supplementing components within the scope of the present disclosure, and such embodiments should also be regarded as falling within the scope of the present disclosure.

| | |
|---|---|
| 10: pedicle screw assembly | 100: screw member |
| 110: screw body | 120: screw head |
| 130: screw thread | 140: screw protrusion |
| 150: screw hollow | 151: first screw hollow |
| 152: second screw hollow | 200: detachment-preventing member |
| 210: detachment-preventing body | 211: detachment-preventing hollow |
| 212: screw head support surface | 213: screw protrusion support surface |
| 220: detachment-preventing ring | 221: detachment-preventing opening |
| 300: rod support member | 310: member-receiving space |
| 320: ring-receiving space | 330: rod-receiving space |
| 340: nut-receiving space | 350: rod support thread |
| 360: rod-coupling opening | 370: protrusion-coupling groove |
| 400: rod support nut | 500: driver support member |
| 510: driver-receiving space | 520: support-member receiving space |
| 530: support-member coupling protrusion | R: rod |
| d: driver | |

1. A pedicle screw assembly comprising:
a screw member extending in one direction and configured to be coupled to a pedicle; and
a detachment-preventing member configured to rotatably support the screw member,
wherein the screw member includes:
a screw body extending in the one direction, the screw body being configured to be at least partially inserted into the pedicle;
a screw head positioned at one end of the screw body in the extending direction, the screw head being configured to be rotatably coupled to the detachment-preventing member about the one direction as an axis; and
a screw protrusion coupled to the screw head opposite to the screw body with the screw head interposed therebetween, the screw protrusion being configured to be rotatably coupled to the detachment-preventing member about the one direction as an axis,
wherein the detachment-preventing member includes:
detachment-preventing body including a first portion rotatably receiving the screw head and a second portion communicating with the first portion and rotatably receiving the screw protrusion, the second portion having a cross-sectional area smaller than that of the one portion.

2. The pedicle screw assembly of claim 1, wherein the detachment-preventing body includes:
a detachment-preventing hollow formed through its interior along the one direction, the detachment-preventing hollow being configured to rotatably receive the screw head and the screw protrusion;
a screw head support surface configured to rotatably support the screw head at one side of the one direction; and
a screw protrusion support surface continuous with the screw head support surface, the screw protrusion support surface being configured to rotatably receive the screw protrusion at an opposite side of the one direction.

3. The pedicle screw assembly of claim 2, wherein the screw head has an outer circumferential surface formed to be at least partially curved, and
the screw head support surface is formed as a curved surface extending roundly to be convex radially outward.

4. The pedicle screw assembly of claim 2, wherein the screw protrusion is configured to have a cross-section with a diameter smaller than a maximum diameter of a cross-section of the screw head; and
the screw protrusion support surface is formed as a curved surface surrounding an outer circumference of the screw protrusion.

5. The pedicle screw assembly of claim 1, comprising:
a rod support member coupled to the detachment-preventing member along the one direction, and configured to allow an external rod to pass through in engagement along a transverse direction; and
a rod support nut coupled to the rod support member along the one direction opposite to the detachment-preventing member with the rod interposed therebetween.

6. The pedicle screw assembly of claim 5, wherein the rod support member includes:
a member-receiving space located inside the rod support member and configured to accommodate the detachment-preventing member;
a rod-receiving space communicating with the member-receiving space and configured to accommodate the rod; and
a nut-receiving space communicating with the rod-receiving space and disposed opposite to the member-receiving space with the rod-receiving space interposed therebetween.

7. The pedicle screw assembly of claim 6, wherein the rod support member includes:
a rod support thread formed on an inner surface of an outer circumference partially surrounding the nut-receiving space, the rod support thread being screw-coupled with a thread formed on an outer circumference of the rod support nut.

8. The pedicle screw assembly of claim 5, wherein the rod support member is coupled to the detachment-preventing member at one side along the one direction and coupled to the rod support nut at an opposite side along the one direction, and
the rod is positioned between the detachment-preventing member and the rod support nut.

9. The pedicle screw assembly of claim 8, wherein the rod support nut is positioned between a first position supporting the rod to bias the rod into abutment with the detachment-preventing member and a second position supporting the rod to keep the rod spaced apart from the detachment-preventing member.

10. The pedicle screw assembly of claim 6, wherein the rod support member includes:
a rod-coupling opening formed through an outer surface radially surrounding both the rod-receiving space and the nut-receiving space, the rod-coupling opening providing an insertion passage for the rod.

11. The pedicle screw assembly of claim 6, wherein the rod support member includes:
a ring-receiving space communicating with the member-receiving space and disposed opposite to the rod-receiving space with the member-receiving space interposed therebetween, and
the detachment-preventing member includes:
a detachment-preventing ring coupled to the screw head opposite to the screw protrusion and accommodated in the ring-receiving space.

12. The pedicle screw assembly of claim 11, wherein the detachment-preventing ring has a hollow formed therein and a detachment-preventing opening recessed on an outer circumference thereof, and
an inner diameter of the detachment-preventing ring is formed to be less than a maximum diameter of a cross-section of the screw head.

13. The pedicle screw assembly of claim 12, wherein the detachment-preventing ring is received in the ring-receiving space after being radially pressed inward so that the detachment-preventing opening is at least partially reduced, and
the detachment-preventing ring received in the ring-receiving space is radially shape-deformed inward so that the detachment-preventing opening is at least partially enlarged.
